(19) United States
(12) Patent Application Publication
(10) Pub. No.: US 2012/0201914 A1
(43) Pub. Date: Aug. 9, 2012

(54) Method and Composition for Improving Animal Oral Health

(75) Inventors: Chifu Huang, Lexington, KY (US); Jeffrey Lee Ebersole, Lexington, KY (US)

(73) Assignee: Chifu Huang, Lexington, KY (US)

(21) Appl. No.: 13/021,767

(22) Filed: Feb. 6, 2011

(51) Int. Cl. A61K 36/42; C11C 3/00; A23K 1/18; A61P 29/00; A23K 1/16; A61K 31/20; A61P 31/04

(52) U.S. Cl. 424/758; 514/560; 554/224; 426/648

(57) ABSTRACT

The invention relates to method and composition to improve the oral health of companion animals targeting the oral health of the animals and directed towards reducing and preventing the buildup of plaque and calculus, leading to periodontal (i.e. gum) disease that contributes to systemic infections that can affect the heart, kidneys, and other tissues. The preparation uses the Lo Han Kuo fruit extract and N-3 fatty acids as bioactive ingredients combined to function as antibacterial and anti-inflammatory agents delivered to the oral cavity of the animals.

### BACKGROUND OF THE INVENTION

Periodontal (gum) disease is a chronic oral bacterial infection that affects virtually all mammalian species. While the bacteria that comprise the ecology of individual animal species differ, certain pathogen species appear to exist across species lines, including humans, nonhuman primates, companion animals, and even animals for commercial uses. As importantly, studies of humans and animals have implicated the chronic oral infection to translocation of bacteria into the systemic circulation and contributing to systemic diseases, such as cardiovascular disease, diabetes, and even adverse pregnancy outcomes. A major change has occurred over the last decade, as research has identified the risk for general health in companion animals that develop periodontal disease, ie infections, in the absence of effective methods to prevent the ravages of the infection. This is particularly pronounced as the animal ages, and while “chew bones/toys”, treats with mechanical cleaning formats, flavored toothpaste for owner provision of care, and professional veterinary dental services have expanded over the last 10 years, these oral infections and disease remain an important problem for pet owners.

Fish oil is widely used as a supplement to improve human diets since it contains the n-3 PUFA, which are essential nutrients for normal physiologic functions. Eicosapentaenoic acid (EPA) and docosahexaenoic acid (DHA) are the major n-3 PUFA in fish oil. N-3 fatty acids refer to a family of unsaturated fatty acids that have a final carbon-carbon double bond in the n-3 position, from the methyl end of the fatty acid. The most widely available source of EPA and DHA for humans comes from cold water oily fish, such as salmon, herring, and mackerel, or purified from algae. Similarly, other mammals cannot synthesize n-3 PUFA de novo, but can form 20- and 22-carbon unsaturated n-3 fatty acids from the eighteen-carbon n-3 fatty acid, α-linolenic acid (ALA). A similar fatty acid, linoleic acid (LA), is an n-6 fatty acid, which has a different bioactivity than α-linolenic acid. Linoleic acid (LA) is used in the cellular biosynthesis of arachidonic acid. As consumer products, n-3 PUFA are usually converted into n-3 fatty acid ethyl esters, which are incorporated in the diets due to the stability of n-3 fatty acid ethyl esters.

EPA and DHA can serve as precursors to eicosanoids which can reduce inflammation. Recent studies have shown that n-3 PUFA can reduce inflammation in the oral cavity of rats and humans with inflammatory periodontitis. Some of these results also demonstrated the effectiveness of an n-3 PUFA-supplemented diet in modulating alveolar bone resorption following P. gingivalis infection, and supported that n-3 fatty acids may be a useful adjunct in the treatment of periodontal disease. While it is well known that n-3 PUFA have anti-inflammatory properties, recent studies show that the three major n-3 PUFA (EPA, DHA, ALA), and their methyl esters EPA-ME, DHA-ME, ALA-ME and ethyl esters EPA-EE, DHA-EE, ALA-EE also possess antibacterial activities. The n-3 PUFA and their ester derivatives exhibit antibacterial activity against various oral pathogens, such as S. mutans, C. albicans, A. actinomycetemcomitans, F. nucleatum, and P. gingivalis. When oral bacteria grow in the oral cavity, and combine with saliva and food debris in the crevices between the tooth and gum, plaque forms and accumulates on the tooth. The dental plaque can result in gingivitis and periodontitis in humans and animals. Therefore, it would appear to be cost-effective to strategically control and prevent the formation of the oral biofilms (i.e. plaque) during the life of the animals.

In addition, sweeteners, such as refined sugar, honey, and high fructose corn syrup are major staples in a broad array of processed animal foods, all of which contribute to bacterial accumulation in the oral cavity. Our recent studies discovered that sweetener Lo Han Kuo extract has anti-microbial activity against oral pathogens. Lo Han Kuo extract has ideal properties as a sweetener suitable for pet and animal foods as it can also inhibit the growth of oral bacteria. In addition, Lo Han Kuo is a low-calorie sweetener, it can help prevent companion animals from becoming obese. Thus, to improve the oral health of animal is to reduce the plaque formation and reduce the amount of oral pathogens which form the biofilm and plaque. The bioactive ingredients, Lo Han Kuo and N-3 fatty acids, are active ingredients for animal foods to protect the build up of plaque and will improve oral health of oral cavity of animals.

### SUMMARY OF THE INVENTION

The invention is related to method and preparation for reducing and/or preventing the buildup of plaque and calculus on the teeth of animals, leading to periodontitis (gum disease) and potential systemic diseases associated with these oral infections. The invention is further directed to animal food products to reduce and/or prevent the buildup of dental plaque and calculus. More specifically, the invention is directed to the use of Lo Han Kuo and N-3 fatty acids either individually or mixed as an additive to pet foods to reduce and/or prevent such buildup. Adding Lo Han Kuo and N-3 fatty acids, either as a liquid or as a dried powder, to animal food products, reduction and/or prevention of dental plaque and calculus can be accomplished. In other words, the invention relates to the use of Lo Han Kuo and N-3 fatty acids as food ingredients. The Lo Han Kuo and N-3 fatty acids can be used by the pet food manufacturer or they can be used by the animal/pet owners as supplements.

In an embodiment, the present invention provides a method for reducing the amount of oral pathogens, which cause gingivitis and periodontitis.

In an embodiment, the present invention provides a method of treating and preventing plaque and calculus build up leading to periodontitis in animals, by incorporating the agents into animal food.

In an embodiment, the present invention provides a nutritional product for providing dental benefits to animals comprising Lo Han Kuo and N-3 fatty acids as bioactive ingredients.

### DETAILED DESCRIPTION OF THE INVENTION

The invention is related to a method for reducing and/or preventing the buildup of plaque and calculus on the teeth of animals. The invention is further directed to animal food products to reduce and/or prevent the buildup of oral bacteria which cause dental plaque and calculus. More specifically, the invention is directed to the use of Lo Han Kuo and N-3 fatty acids either by itself or as an additive to pet food to reduce and/or prevent such buildup. By adding Lo Han Kuo and N-3 fatty acids, either as a liquid or as a dried powder, to a domestic animal food product, reduction and/or prevention of dental plaque and calculus can be accomplished. In other words, the invention relates to the use of Lo Han Kuo and N-3 fatty acids as food ingredients. The Lo Han Kuo and N-3 fatty acids can be used by pet food manufacturers or they can be provided by pet owners. The Lo Han Kuo and N-3 fatty acids can be added to the pet food by the pet food manufacturer or it can be added by the pet owner in an amount effective to reduce or prevent the accumulation of plaque and calculus, leading to periodontitis. The Lo Han Kuo and N-3 fatty acids may further be used in a method of reducing the incidence of bad breath both cats and dogs, or of reducing or preventing gingivitis and other periodontal diseases.

The following examples are presented by way of illustration, not of limitation.

### EXAMPLE 1

As nutritional supplement, 1% Lo Han Kuo extract and 1% N-3 fatty acids are incorporated into dry animal food pellets, or kibble, or pet snacks to serve animal. The concentration of Lo Han Kuo extract can be in the range of 0.1%-5%, and that of N-3 fatty acids can be in the range of 0.1-5%, but not limited, where N-3 fatty acids are N-3 fatty acids (Eicosapentaenoic acid (EPA) and docosahexaenoic acid (DHA), α-linolenic acid (ALA) used individually or mixed.

### EXAMPLE 2

Lo Han Kuo extract and N-3 fatty acids are used to make a novel pet treat. For instance, mix 2 cups oats, 2 cups wheat bran, 1 cup cooked liver, ½ cup vegetable oil, 1% Lo Han Kuo extract and 1% N-3 fatty acids are mixed and stir well. A single spoonful can be shaped and baked in a 350° F. oven for 10-15 minutes until the treats are browned. The treat is then ready to serve dog or cat. Both the LHK extract and fatty acids are stable at this heating temperature.

### EXAMPLE 3

For the Lo Han Kuo extract and N-3 fatty acids pet treat ½ cup dry milk, ½ cup wheat germ, one teaspoon of Lo Han Kuo extract, one teaspoon N-3 fatty acids, one 3⅓ oz jar of strained liver baby food or homemade blended liver, are mixed. Small balls of the mixture are produced and flattened onto a cookie sheet. Bake at 350° F. for 8-10 minutes. These can be stored in refrigerator bags, or for extended times in freezer bags. The treat is ready to serve dog or cat. Both the LHK extract and fatty acids are stable at these heating and cooling temperatures.

### BEST MODE FOR CARRYING OUT THE INVENTION

The preferred embodiments of this invention are described herein, which includes the best mode that is known to the inventor for implementing the invention. Even though the invention has been described with reference to particular embodiments, it is to be understood that these embodiments are merely illustrative of the applications and principles of the invention. Therefore, it is to be understood that many modifications may be made to the illustrative embodiments without departing from the spirit and the scope of the invention as defined in the attached claims.

### INDUSTRIAL APPLICABILITY

This invention technology is capable of producing compositions that can be used in a variety of applications and products where the animal oral health can be improved. The compositions can be used as a dietary/nutritional supplement to a standard diet of animals, including cats, dogs, cows, pigs, horses, but not limited to these animals.

In addition, the compositions can also be used in the form of food such as kibble, pet snacks, pet treats, or as powder or liquid provided separately or added to the normal diet.

1. A method to improve the oral health of companion animals comprised of delivering to the oral cavity of the animal in foodstuffs, treats, dietary supplement, but not limited to, containing antibacterial and anti-inflammatory levels of the ingredients, Lo Han Kuo (Siraitia grosvenori) fruit extract and N-3 fatty acids (Eicosapentaenoic acid (EPA) and docosahexaenoic acid (DHA), α-linolenic acid (ALA).

2. A preparation to improve the oral health of companion animals comprised of adding the ingredient N-3 fatty acids to the animals foodstuffs, treats, or dietary supplements to provide antibacterial actions against oral bacteria.

3. A preparation to improve the oral health of companion animals comprised of adding the ingredients N-3 fatty acids to the animals foodstuffs, treats, or dietary supplements to provide anti-inflammatory therapy to improve oral health.

4. A preparation to improve the oral health of companion animals comprised of adding the ingredients of the anti-cariogenic sweetener Lo Han Kuo to the animal foodstuffs, treats, or dietary supplements to provide enhance the palatability of the materials and provide additional antibacterial targeting for oral bacteria.

5. The preparation is directed to the use of Lo Han Kuo and N-3 fatty acids either individually or mixed as additive ingredients.

6. The preparation is directed to the use of Lo Han Kuo and N-3 fatty acids either individually or mixed as additive ingredients, with addition of vitamin E, C, and D.

7. The product can be used as a dietary/nutritional supplement to a standard diet of animals, including cats, dogs, cows, pigs, horses, but not limited to these animals.

8. The products can be in the form of food such as kibble, pet snacks, pet treats, or as powder or liquid provided separately or added to the normal diet.
